(19) United States
(12) Patent Application Publication
(10) Pub. No.: US 2026/0256447 A1
(43) Pub. Date: Sep. 3, 2026

(54) BREAST ULTRASOUND IMAGING METHOD AND SYSTEM, AND NON-TRANSITORY COMPUTER-READABLE MEDIUM

(71) Applicant: GE Precision Healthcare LLC, Waukesha, WI (US)

(72) Inventors: Liye Pei, Wuxi (CN); Chunyan Qi, Wuxi (CN)

(21) Appl. No.: 19/554,346

(22) Filed: Mar. 2, 2026

(30) Foreign Application Priority Data
Feb. 28, 2025 (CN) 202510241380.6

(51) Int. Cl. A61B 8/08; A61B 8/00; G06T 7/00; G06T 7/11; G06T 7/174

(52) U.S. Cl. CPC A61B 8/0825; A61B 8/461; A61B 8/483; G06T 7/0014; G06T 7/11; G06T 7/174; G06T 2207/10136; G06T 2207/20084; G06T 2207/30068; G06T 2207/30096; G06T 2207/30196; G06T 2207/30204

(57) ABSTRACT

A breast ultrasound imaging method, including: acquiring a volumetric ultrasound image related to the breast, wherein the volumetric ultrasound image comprises a plurality of two-dimensional images in a depth direction; identifying a nipple region in each two-dimensional image; determining a nipple region in the volumetric ultrasound image and generating a corresponding nipple marker; comparing the nipple region in the volumetric ultrasound image with the nipple region in each two-dimensional image, and automatically selecting a two-dimensional image at a first depth according to a comparison result; and displaying the two-dimensional image at the first depth and the nipple marker simultaneously.

### CROSS-REFERENCE TO RELATED APPLICATION

This application claim priority to Chinese Patent Application No. 202510241380.6, which was file on February 28, 2025 at the Chinese Patent Office. The entire contents of the above-listed application are incorporated by reference herein in their entirety.

### TECHNICAL FIELD

The present application relates to the field of ultrasound imaging, and in particular, to a method and a system for performing ultrasound imaging on a breast.

### BACKGROUND

Ultrasound imaging is a real-time, non-invasive imaging technology suitable for imaging of different organs. For example, automatic breast ultrasound imaging is one of the ultrasound imaging techniques that is suitable for high-quality imaging of the breast. Automatic breast ultrasound imaging is capable of collecting and processing volumetric ultrasound data related to a breast to generate a volumetric ultrasound image of the breast. Furthermore, the volumetric ultrasound image may be segmented into a plurality of coronal plane two-dimensional images in a depth direction (which may be understood as a direction from the skin to the interior of the body), making it convenient for a doctor to observe the coronal plane images at different depths and determine a region of interest, such as a lesion, therein.

Automatic breast ultrasound imaging generally provides an automatic marking function for a nipple position. A nipple marker can be used as a reference point for the doctor to record the position of a region of interest. For example, after a suspicious lesion is found in a breast ultrasound image, the doctor can record the suspicious lesion according to the position of the suspicious lesion relative to the nipple marker. Thus, the accuracy of the nipple marker is significantly important.

### SUMMARY OF THE INVENTION

Some embodiments of the present application provide a breast ultrasound imaging method, comprising: acquiring a volumetric ultrasound image related to the breast, wherein the volumetric ultrasound image comprises a plurality of two-dimensional images in a depth direction; identifying a nipple region in each two-dimensional image; determining a nipple region in the volumetric ultrasound image and generating a corresponding nipple marker; comparing the nipple region in the volumetric ultrasound image with the nipple region in each two-dimensional image, and automatically selecting a two-dimensional image at a first depth according to a comparison result; and displaying the two-dimensional image at the first depth and the nipple marker simultaneously.

Optionally, the nipple marker is applied to each two-dimensional image, and the position of the nipple marker in each two-dimensional image is consistent.

Optionally, the acquisition of a volumetric ultrasound image of the breast comprises: moving an ultrasonic transducer on a plane perpendicular to the depth direction while using the ultrasonic transducer to perform two-dimensional ultrasound imaging on the breast to obtain an image set related to the breast; and performing synthesis processing on the image set to obtain the volumetric ultrasound image.

Optionally, the plurality of two-dimensional images are acquired by segmenting the volumetric ultrasound image.

Optionally, the method further comprises: generating and displaying a depth marker, wherein the depth marker indicates the first depth.

Optionally, the method further comprises: in response to being operated, moving the depth marker to indicate a second depth, and displaying a two-dimensional image at the second depth.

Optionally, the comparison of the nipple region in the volumetric ultrasound image with the nipple region in each two-dimensional image comprises: respectively comparing the nipple region in each two-dimensional image with the nipple region in the volumetric ultrasound image to obtain similarity therebetween; and the automatically selecting a two-dimensional image at a first depth according to a comparison result comprises: automatically selecting a two-dimensional image with high similarity as the two-dimensional image at the first depth according to a similarity comparison result.

Optionally, the identification of a nipple region in each two-dimensional image is implemented by means of an artificial neural network.

Optionally, the determination of a nipple region in the volumetric ultrasound image comprises: performing mask processing on each two-dimensional image to obtain a plurality of nipple mask images; processing the plurality of nipple mask images to obtain a final nipple mask image; and determining a nipple region in the final nipple mask image as the nipple region in the volumetric ultrasound image.

Optionally, the processing of the plurality of nipple mask images to obtain a final nipple mask image comprises: performing screening on the plurality of nipple mask images, wherein the screening is performed according to at least one of the size and the position of a nipple region in each nipple mask image; determining an overlapping region of nipple regions in a plurality of nipple mask images obtained after screening; and determining the final nipple mask image based on the overlapping region.

Some other embodiments of the present application further provide a breast ultrasound imaging system, comprising a processor. The processor is configured to perform any of the foregoing methods.

Optionally, the system further comprises: an ultrasonic transducer, wherein under control of the processor, the ultrasonic transducer emits an ultrasonic beam toward the breast and receives an echo signal; and a display, wherein the display performs displaying under control of the processor.

Some other embodiments of the present application further provide a non-transitory computer-readable medium. The non-transitory computer-readable medium has a computer program stored thereon, the computer program has at least one code segment, and the at least one code segment is executable by a machine to cause the machine to perform the steps of any of the foregoing methods.

It should be understood that the brief description above is provided to introduce, in a simplified form, concepts that will be further described in the detailed description. The brief description above is not meant to identify key or essential features of the claimed subject matter. The scope is defined uniquely by the claims that follow the detailed description. Furthermore, the claimed subject matter is not limited to implementations that solve any deficiencies raised above or in any section of the present disclosure.

### BRIEF DESCRIPTION OF THE DRAWINGS

The present application will be better understood by reading the following description of non-limiting embodiments with reference to the accompanying drawings, where:

FIG. 1 is a perspective view of a breast ultrasound imaging system according to some embodiments;

FIG. 2 is a block diagram of various system components of a breast ultrasound imaging system according to some embodiments of the present application;

FIG. 3 is a schematic diagram of a breast ultrasound imaging method according to some embodiments of the present application;

FIG. 4 is a schematic diagram of a two-dimensional image with a nipple marker applied thereto according to an embodiment of the present application;

FIG. 5 is a schematic diagram of a two-dimensional image with a nipple marker applied thereto according to another embodiment of the present application;

FIG. 6 is a schematic diagram of a depth selection method for a two-dimensional image according to some embodiments of the present application;

FIG. 7 is a schematic diagram of processing a volumetric ultrasound image according to some embodiments of the present application; and

FIG. 8 is a schematic diagram of an ultrasound image including a depth marker according to some embodiments of the present application.

### DETAILED DESCRIPTION

In some cases, positions of automatically generated nipple markers may not match actual nipple positions in two-dimensional images at certain depths. This may be because the nipple markers are determined by performing comprehensive computation according to nipple positions in an entire depth direction during automatic generation. This result does not ensure a good match with a nipple position in a two-dimensional image at each depth. Although this generally does not cause problems in auxiliary diagnosis, it can greatly affect the doctor's confidence in diagnosis. For example, when an ultrasound apparatus displays a two-dimensional image at a certain depth, a nipple position in the image may just deviate from the position of a nipple marker, and after observation, it is difficult for the doctor to determine whether the deviation is caused by a failure in automatic marking or a normal error. The doctor needs to further perform manual operations to confirm, for example, the doctor needs to check a matching degree between nipple positions and nipple markers at different depths. This affects the doctor's confidence in diagnosis and reduces the efficiency of ultrasound imaging.

Specific embodiments of the present application are described below. It should be noted that in the specific description of these embodiments, for a concise description, the present application may not describe in detail all of the features of the actual embodiments. It should be understood that in the actual implementation process of any implementation, just as in the process of any one engineering project or design project, a variety of specific decisions are often made to achieve specific goals of the developer and to meet system-related or business-related constraints, which may also vary from one implementation to another. Furthermore, it should also be understood that although efforts made in such development processes may be complex and extended, for a person of ordinary skill in the art related to the disclosure of the present application, some design, manufacture or production changes made on the basis of the technical disclosure of the present disclosure are only conventional technical means, and should not be construed that the content of the present disclosure is insufficient.

Unless otherwise defined, the technical or scientific terms used in the claims and the description should be as they are usually understood by those possessing ordinary skill in the technical field to which they belong. The terms “first”, “second” and similar words used in the present application and the claims do not express any order, quantity or importance, but are merely intended to distinguish between different constituents. The terms “one” or “a/an” and similar terms do not express a limitation of quantity, but rather that at least one is present. The terms “include” or “comprise” and similar words indicate that an element or object preceding the terms “include” or “comprise” encompasses elements or objects and equivalent elements thereof listed after the terms “include” or “comprise”, and do not exclude other elements or objects. The terms “connect” or “link” and similar words are not limited to physical or mechanical connections, and are not limited to direct or indirect connections.

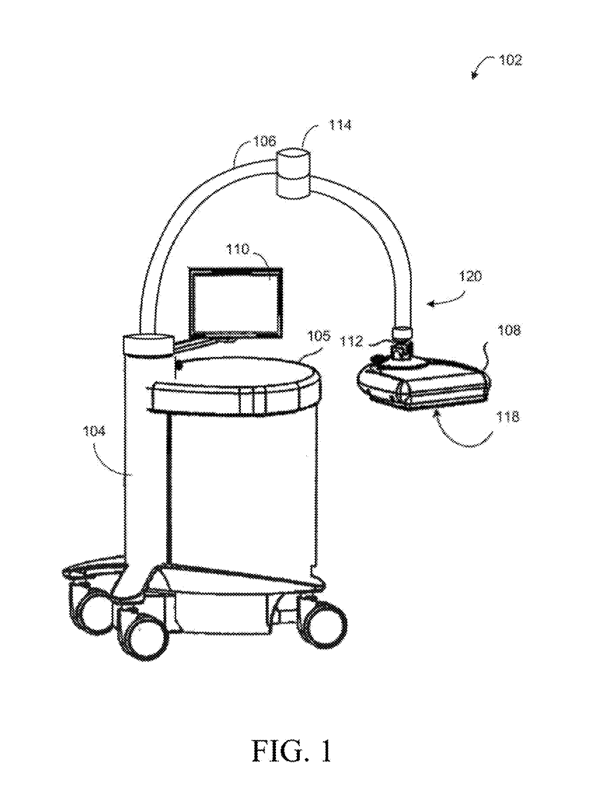

FIG. 1 shows a perspective view of a breast ultrasound imaging system 102 according to some embodiments. An automatic breast ultrasound imaging apparatus is used as an example for exemplary description. The body of the breast ultrasound imaging system 102 may include a main device, a display 110, an adjustable arm 106, and a scanning assembly 108. The main device may include a body frame 104, an ultrasonic processor housing 105, and an ultrasonic processor inside the housing 105. The specific structure of each component will be illustrated in detail below.

The body frame 104, the ultrasonic processor housing 105 containing the ultrasonic processor, a movable and adjustable support arm (for example, an adjustable arm) 106 including a hinge joint 114, the scanning assembly 108 connected to a first end 120 of the adjustable arm 106 by means of a ball and socket connector (for example, a ball joint) 112, and the display 110 connected to the body frame 104. The display 110 is connected to the body frame 104 at a joining point where the adjustable arm 106 enters the body frame 104. Since the display 110 is directly connected to the body frame 104 rather than the adjustable arm 106, the display 110 does not affect the weight of the adjustable arm 106 and a balancing mechanism of the adjustable arm 106. In one example, the display 110 is rotatable in horizontal and transverse directions (for example, rotatable around a central axis of the body frame 104), but is not vertically movable. In an alternative example, the display 110 may also be vertically movable. Although FIG. 1 illustrates the display 110 connected to the body frame 104, in other examples, the display 110 may be connected to different components of the breast ultrasound imaging system 102, such as, connected to the ultrasonic processor housing 105, or positioned away from the breast ultrasound imaging system 102.

In one embodiment, the adjustable arm 106 is configured and adapted such that the pressing/scanning assembly 108 (i) is neutrally buoyant in space, or (ii) has a light net downward weight (for example, 1-2 kg) for pressing the breast, while allowing easy user operation. In an alternative embodiment, the adjustable arm 106 is configured such that the scanning assembly 108 is neutrally buoyant in space during positioning of a scanner on tissue of a patient. Then, after the scanning assembly 108 is positioned, internal components of the breast ultrasound imaging system 102 may be adjusted to apply a desired downward weight for pressing the breast and improved image quality. In one example, the downward weight (for example, a force) may be in the range of 2-11 kg.

As described above, the adjustable arm 106 includes the hinge joint 114. The hinge joint 114 divides the adjustable arm 106 into a first arm portion and a second arm portion. The first arm portion is connected to the scanning assembly 108 and the second arm portion is connected to the body frame 104. The hinge joint 114 allows the second arm portion to rotate relative to the second arm portion and the body frame 104. For example, the hinge joint 114 allows the scanning assembly 108 to translate transversely and horizontally, but not vertically, relative to the second arm portion and the body frame 104. In such manner, the scanning assembly 108 can rotate toward the body frame 104 or away from the body frame 104. However, the hinge joint 114 is configured to allow the entire adjustable arm 106 (for example, the first arm portion and the second arm portion) to move vertically together as a whole (for example, translating upward and downward along with the body frame 104).

The scanning assembly 108 may include a film assembly 118 having a film that is in a substantially tensioned state to be at least partially attached, for pressing the breast. The film assembly 118 has a bottom surface for contacting the breast, and when the bottom surface is in contact with the breast, the transducer sweeps over a top surface of the film to scan the breast. In one example, the film is a tensioned fabric sheet.

The film assembly 118 may further include an outer frame and a film. The film is fixedly disposed in the outer frame, and the outer frame is detachably connected to the scanning assembly. In an ultrasonic imaging process performed by the ultrasonic imaging system, one side surface of the film can be at least partially in contact with an ultrasonic transducer, and another side surface of the film is at least partially in contact with a tissue to be scanned. Such an arrangement can ensure that the ultrasonic transducer transmits and receives signals with less attenuation, and can fix the breast to be scanned to facilitate scanning.

Optionally, the adjustable arm may include a potentiometer (not shown) to allow position and direction sensing performed by the pressing/scanning assembly 108, or may use other types of position and direction sensing (such as gyroscope, magnetic, optical, and radio frequency (RF)). A fully functional ultrasonic engine may be provided within the ultrasonic processor housing 105, and is configured to drive the ultrasonic transducer, and generate volumetric breast ultrasound data from a scan in conjunction with related position and orientation information. In some examples, volumetric scan data may be transmitted to another computer system by using any of a variety of data transmission methods known in the art for further processing, or the volumetric scan data may be processed by the ultrasound engine. A general-purpose computer/processor integrated with the ultrasound engine may further be provided for general user interface and system control. The general-purpose computer may be a self-contained stand-alone unit, or may be remotely controlled, configured, and/or monitored by remote stations connected across networks.

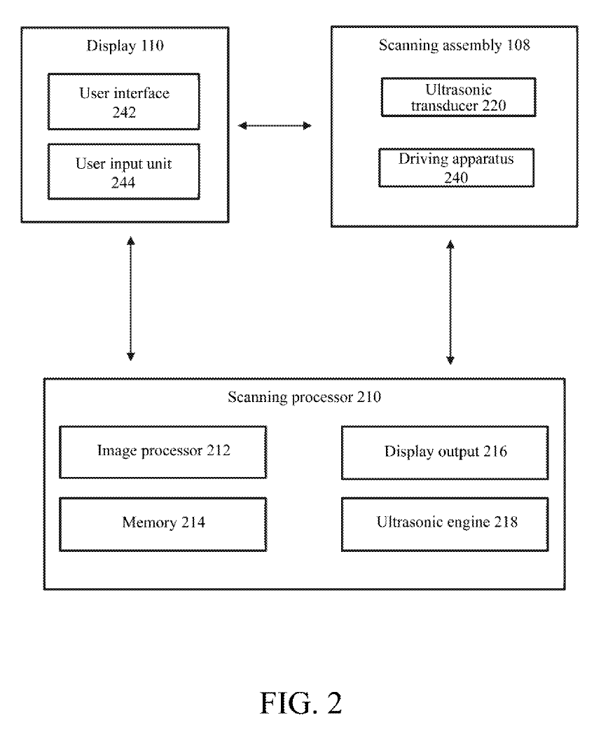

Reference is made to FIGS. 1 and FIG. 2 together. FIG. 2 is a block diagram that schematically shows various system components of the breast ultrasound imaging system 102, including the scanning assembly 108, the display 110, and a scanning processor 210. In an example, the scanning processor 210 may be included within the ultrasonic processor housing 105 of the breast ultrasound imaging system 102. As shown in the embodiment of FIG. 2, the scanning assembly 108, the display 110, and the scanning processor 210 are separate components in communication with each other. However, in some embodiments, one or more of these components may be integrated (for example, the display and the scanning processor may be included in a single component).

In the example of FIG. 2, the scanning assembly 108 includes at least an ultrasonic transducer 220 and a driving apparatus 240. The ultrasonic transducer 220 includes a transducer array of transducer elements, such as piezoelectric elements, which convert electrical energy into ultrasonic waves and then detect reflected ultrasonic waves.

The scanning assembly 108 may communicate with the scanning processor 210 to send raw scan data to an image processor. The scanning assembly 108 may optionally communicate with the display 110 to notify a user to reposition the scanning assembly as described above, or to receive information from the user (via a user input unit 244).

In the example of FIG. 2, the scanning processor 210 includes an image processor 212, a memory 214, display output 216, and an ultrasonic engine 218. The ultrasonic engine 218 may drive activation of the transducer elements of the ultrasonic transducer 220, and in some embodiments, the driving apparatus 240 may be activated. Furthermore, the ultrasonic engine 218 may receive raw image data (for example, ultrasonic echoes) from the scanning assembly 108. The raw image data may be sent to the image processor 212 and/or a remote processor (for example, via a network) and be processed to form a displayable image of a tissue sample. It should be understood that in some embodiments, the image processor 212 may be included in the ultrasonic engine 218.

Information may be transmitted from the ultrasonic engine 218 and/or the image processor 212 to the user of the breast ultrasound imaging system 102 via the display output 216 of the scanning processor 210. In one example, the user of the ultrasound imaging system may include an ultrasonic technician, a nurse, or a physician such as a radiologist. For example, a processed image of scanned tissue may be sent to the display 110 via the display output 216. In another example, information (such as the progress of scanning) related to parameters of the scanning may be sent to the display 110 via the display output 216. The display 110 may include a user interface 242 configured to display images or other information to the user. Furthermore, the user interface 242 may be configured to receive an input from the user (such as by means of a user input unit 244), and send the input to the scanning processor 210. In one example, the user input unit 244 may be a touch screen of the display 110. However, other types of user input mechanisms are also possible, such as a mouse, a keyboard, and the like.

The scanning processor 210 may further include the memory 214. The memory 214 may include movable and/or permanent devices, and may include an optical memory, a semiconductor memory, and/or a magnetic memory, etc. The memory 214 may include a volatile, non-volatile, dynamic, static, read/write, read only, random access, sequential access, and/or annex memory. The memory 214 may store non-transitory instructions executable by a controller or processor (such as a controller 218 or the image processor 212) to perform one or more methods or routines as described below. The memory 214 may store raw image data received from the scanning assembly 108, processed image data received from the image processor 212 or the remote processor, and/or additional information.

Automatic breast ultrasound imaging generally provides an automatic marking function for a nipple position. The nipple marker can be used as a reference point for the doctor to record the position of a region of interest, such as a lesion. Such a nipple marker may be used for simultaneous display with the image data. In some cases, a nipple position in an image that is being displayed may just deviate from the position of the nipple marker, and after observation, it is difficult for the doctor to determine whether the deviation is caused by a failure in automatic marking or a normal error. The doctor needs to further perform manual operations to confirm, for example, the doctor needs to check a matching degree between nipple positions and nipple markers at different depths. This affects the doctor's confidence in diagnosis and reduces the efficiency of ultrasound imaging. In view of this, improvements are provided in one or more embodiments of the present application.

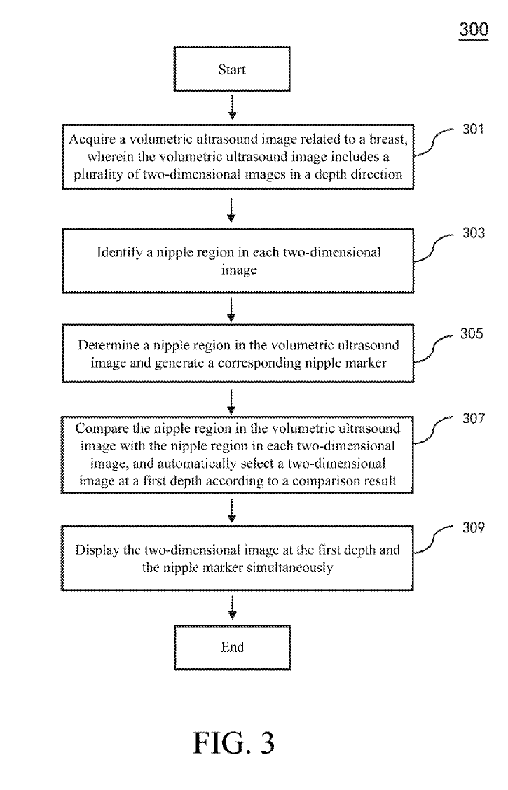

Referring to FIG. 3, some embodiments of the present application provide a breast ultrasound imaging method 300. The method 300 includes:

step 301, acquiring a volumetric ultrasound image related to the breast, wherein the volumetric ultrasound image includes a plurality of two-dimensional images in a depth direction;

step 303, identifying a nipple region in each two-dimensional image;

step 305, determining a nipple region in the volumetric ultrasound image and generating a corresponding nipple marker;

step 307, comparing the nipple region in the volumetric ultrasound image with the nipple region in each two-dimensional image, and automatically selecting a two-dimensional image at a first depth according to a comparison result; and

step 309, displaying the two-dimensional image at the first depth and the nipple marker simultaneously.

The foregoing method can ensure that a two-dimensional image with the best consistency between the nipple position and the nipple marker among the plurality of two-dimensional images in the depth direction is positioned and displayed. In this way, when the user views the two-dimensional image, a correspondence relationship between the nipple position and the nipple marker in the two-dimensional image can be more accurately determined. Furthermore, the foregoing method of the present application can ensure that the correspondence relationship is good. Therefore, this avoids a decrease in diagnosis confidence and additional work in a work flow caused due to discrepancy in a positional relationship between a nipple region and a nipple marker in a two-dimensional image at a certain depth displayed by default.

It may be understood that the foregoing method 300 may be performed by a processor, for example, may be implemented by a processor in the breast ultrasound imaging system described in any of the embodiments of FIGS. 1 and FIG. 2 of the present application by executing a specific algorithm.

The acquisition of a volumetric ultrasound image related to the breast may be implemented by the processor controlling the driving apparatus 240 to drive the ultrasonic transducer 220. In an example, the processor obtains an image set related to the breast by moving the ultrasonic transducer 220 on a plane perpendicular to the depth direction while using the ultrasonic transducer 220 to perform two-dimensional ultrasound imaging on the breast. Furthermore, synthesis processing is performed on the image set to obtain the volumetric ultrasound image.

It may be understood that the volumetric ultrasound image described above in the present application may be ultrasound images that are related to an entire breast and that are obtained by scanning with the breast ultrasound imaging system. However, in another embodiment, the volumetric ultrasound image may alternatively be a subset of the ultrasound images of the entire breast, for example, a volumetric ultrasound image within a certain depth range. The depth range may be preset, and a selection manner may be based on an empirically determined depth range within which a nipple is located, without processing a volumetric ultrasound image outside the depth range. Therefore, an amount of data computation can be reduced as much as possible while meeting the effects achieved by the embodiments of the present application.

Each image in an image set obtained through a plain scan may be understood as a two-dimensional image extending in the depth direction. Correspondingly, a volumetric ultrasound image obtained after the image set is synthesized also includes image information in the depth direction. That is, the volumetric ultrasound image includes a plurality of two-dimensional images in the depth direction.

The plurality of two-dimensional images may be obtained by segmenting the volumetric ultrasound image. For example, the ultrasound imaging system can segment the volumetric ultrasound image, by default, into a certain quantity of two-dimensional images in the depth direction. In another example, a specific quantity may be customized by the user. It may be understood that although the images are two-dimensional images, each two-dimensional image is allowed to have a certain thickness. The thickness may be freely configured. For example, a specific thickness may be determined according to factors such as an overall depth of the volumetric ultrasound image and precision of diagnosis and treatment required. The present application does not make a limitation.

In some embodiments, a nipple region in each two-dimensional image may be identified in various manners. For example, identification of a nipple region may be implemented by means of an artificial neural network. In a non-limiting embodiment, the artificial neural network may be divided into two or more than two layers, such as an input layer for receiving an input image, an output layer for outputting an output image, and/or one or more intermediate layers. Layers of a neural network represent different groups or sets of artificial neurons, which may represent different functions. The different functions are executed by the processor on each two-dimensional image to identify features contained therein. Artificial neurons in a layer of the neural network may examine individual pixels in an input two-dimensional image. The artificial neurons use different weights in a function applied to the input two-dimensional image, so as to attempt to identify an object (for example, the nipple region) therein. The neural network produces an identification result for the nipple region by assigning or associating different pixels in an output image with different anatomical features, on the basis of analysis of pixel characteristics. It should be noted that the foregoing description of the artificial neural network is only an exemplary description, and the present disclosure is not limited thereto. In addition, in addition to using artificial intelligence technologies such as artificial neural networks, any other technologies in the prior art may also be used to identify the nipple region, which will not be repeated.

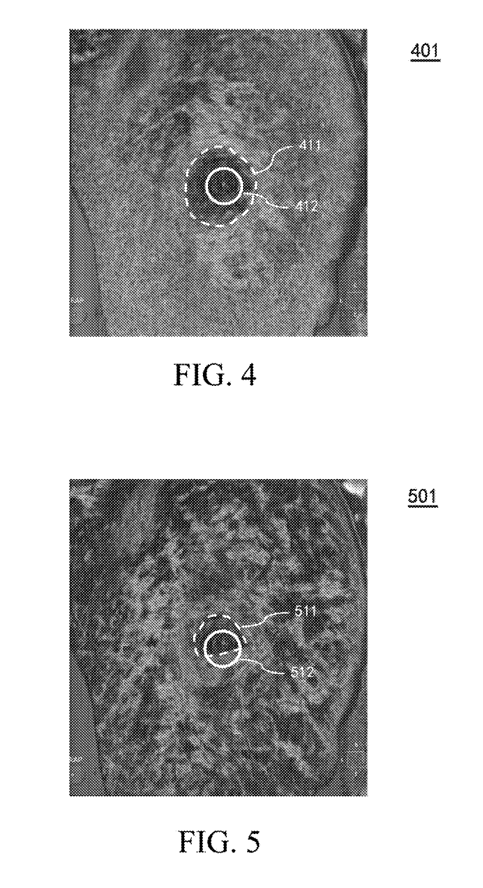

It may be understood that, from an anatomical point of view, the position of the nipple in the depth direction substantially extends vertically. Correspondingly, to keep consistent with the human physiological structure, in some embodiments of the present application, the nipple marker is applied to each two-dimensional image, and the position of the nipple marker in each two-dimensional image is consistent. Detailed description is further provided with reference to FIGS. 4 and FIG. 5 of the present application. FIG. 4 is a schematic diagram of a two-dimensional image with a nipple marker applied thereto according to an embodiment of the present application. FIG. 5 is a schematic diagram of a two-dimensional image with a nipple marker applied thereto according to another embodiment of the present application.

As shown in FIGS. 4 and FIG. 5, a two-dimensional image 401 and a two-dimensional image 501 respectively represent coronal plane two-dimensional images at different depths. The two images respectively include a first nipple region 411 and a first nipple region 511. It can be seen that the first nipple region 411 and the second nipple region 511 at different depths are different in size and position due to the human physiological structure. Furthermore, nipple markers 412 and 512 at the same positions are respectively applied to the two-dimensional images 401 and 501. The same positions may be understood as being aligned in the depth direction (that is, in a direction perpendicular to a coronal plane). Correspondingly, the nipple markers 412 and 512 appear at the same position on a plane of the two-dimensional images. Therefore, when the doctor views two-dimensional ultrasound images at different depths, the position of the nipple marker does not shift.

Still referring to FIGS. 4 and FIG. 5, it can be seen by observing relative positions of the nipple region 411 and the nipple marker 412 that there is a good correspondence relationship between the nipple region 411 and the nipple marker 412. The nipple marker 412 is properly positioned at a relatively central position of the nipple region 411. It can be seen by observing relative positions of the nipple region 511 and the nipple marker 512 that a deviation occurs in a correspondence relationship between the nipple region 511 and the nipple marker 512. Specifically, although the nipple region 511 and the nipple marker 512 overlap to some extent, a lower right portion of the nipple marker 512 has already exceeded the range of the nipple region 511. In this case, if a coronal plane two-dimensional image is being used for display to the doctor, and a default coronal plane image displayed is the two-dimensional image 501, it will inevitably cause trouble to the doctor. It will be difficult for the doctor to determine whether the foregoing discrepant result is a special case in the current depth range (if yes, it is acceptable) or an unqualified nipple marker due to other reasons (in this case, it is unacceptable). In this case, the doctor will have to check two-dimensional images at a plurality of depths to determine consistency between the nipple position and the nipple marker therein, and finally make a determination.

After the solution described in any of the foregoing embodiments of the present application is used, the foregoing problem can be effectively resolved. According to the embodiments of the present application, the two-dimensional image selected for display has been subject to comparison and screening Therefore, it can be ensured that the nipple region and the nipple marker in the two-dimensional image have good consistency. For example, it is assumed that the two-dimensional image used for display is the two-dimensional image 401 automatically selected after determination with an algorithm. In this case, there is a good correspondence relationship between the nipple region 411 and the nipple marker 412 in the two-dimensional image 401. After seeing such an image, the doctor is able to build diagnostic confidence without having to determine whether the nipple marker is qualified, thereby simplifying a work flow.

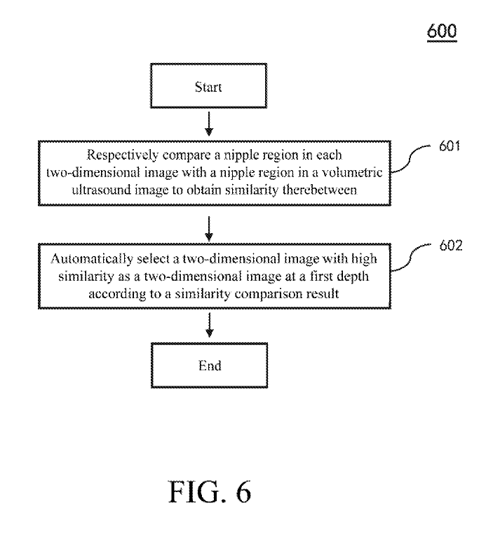

As described above in the present application, a manner of determining a two-dimensional image for display may be: comparing the nipple region in the volumetric ultrasound image with the nipple region in each two-dimensional image, and automatically selecting a two-dimensional image at a first depth according to a comparison result. An exemplary description of a comparison method will be provided below. Referring to FIG. 6, FIG. 6 shows a schematic diagram of a depth selection method 600 for a two-dimensional image according to some embodiments of the present application.

In step 601, the nipple region in each two-dimensional image is compared with the nipple region in the volumetric ultrasound image respectively to obtain similarity therebetween; and

in step 602, a two-dimensional image with high similarity is automatically selected as the two-dimensional image at the first depth according to a similarity comparison result.

The inventor has realized that the nipple marker is generated according to the position of the nipple region in an overall image (a volumetric image). Therefore, by comparing nipple regions in respective two-dimensional images at different depths with a nipple region in the overall image, similarity between the two-dimensional images at different depths and the nipple marker can be determined through comparison. Therefore, in a two-dimensional image having a nipple region with high similarity to the nipple region in the volumetric ultrasound image, the nipple region also has high similarity to the nipple marker. Positioning and displaying the two-dimensional image at this depth can, to the maximum extent possible, enable the doctor to visually view an image with good correspondence between the nipple region and the nipple marker, enhancing the confidence in diagnosis.

A manner of determining high similarity may be given based on understanding of a person of ordinary skill in the art. An exemplary description is provided below. In an embodiment, nipple regions in the two-dimensional image and the volumetric image may be compared to obtain an overlap ratio therebetween, and the higher the overlap ratio is, the higher the similarity is considered to be. In addition, a similarity threshold may be set. A two-dimensional image with similarity not lower than the threshold may be identified as an image with high similarity. In another embodiment, there may be a plurality of images with high similarity, and the processor may automatically select one among the images with high similarity for display. In addition, a two-dimensional image with the highest similarity may be identified as a two-dimensional image with high similarity and used for display. The foregoing will not be further enumerated.

As described above in the present application, the nipple region in the two-dimensional image may be identified in a plurality of different manners. Similarly, there may be various manners to determine the nipple region in the volumetric ultrasound image and generate a corresponding nipple marker. An exemplary description is provided below.

In an example, the nipple region in the volumetric ultrasound image may also be implemented by means of artificial intelligence, for example, may be identified with reference to the configuration manner of the artificial neural network described above in the present application. After identification, a corresponding nipple marker can be generated for an identified nipple region. The nipple marker may be configured as a circular marker and positioned at a suitable position, such as the center of the nipple region. It may be understood that the nipple marker may be in other shapes and positioned in other manners, as long as it is convenient for the user to observe and a spatial correspondence relationship with the nipple region in the volumetric ultrasound image can be shown. The present application does not make a unique definition.

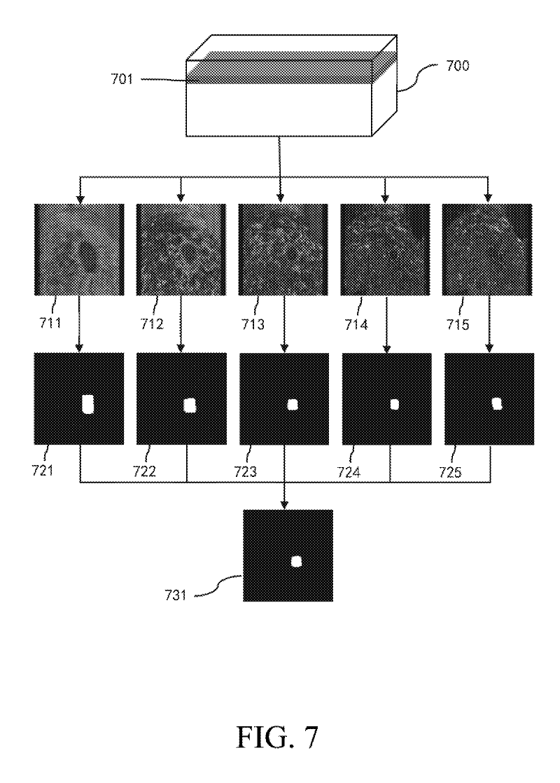

In another example, this may alternatively be implemented by using other algorithms, such as a masking algorithm. The following provides an exemplary description. Referring to FIG. 7, FIG. 7 shows a schematic diagram of processing a volumetric ultrasound image according to some embodiments of the present application.

As shown in FIG. 7, an overall volumetric ultrasound image 700 may include a volumetric ultrasound image 701. The overall volumetric ultrasound image 700 may be obtained by using the ultrasonic transducer of the breast ultrasound imaging system to acquire and synthesize an image set in a direction parallel to a coronal plane as described above in the present application. The volumetric ultrasound image 701 may be a subset of the overall volumetric ultrasound image 700, and the subset includes a depth range within which all nipples are located. Performing processing described below only on the volumetric ultrasound image 701 helps to reduce an amount of data computation. However, it may be understood that in another example, the processing described below may alternatively be performed on the overall volumetric ultrasound image 700.

The volumetric ultrasound image 701 may include a plurality of two-dimensional images 711 to 715 in the depth direction. For a manner for acquiring the two-dimensional images 711 to 715, reference is made to the foregoing description in the present application, and the manner will not be repeated herein. However, it should be noted that FIG. 7 is only an example showing a case of five two-dimensional images 711 to 715, but the solution of the present application is not limited thereto, and there may also exist a case of less than or more than five two-dimensional images. In addition, the nipple regions in the plurality of two-dimensional images 711 to 715 can be identified by using an algorithm such as an artificial neural network.

Furthermore, mask processing may be performed on each of the plurality of two-dimensional images 711 to 715 to obtain a plurality of nipple mask images 721 to 725, as shown in FIG. 7. For mask processing, reference may be made to any manner in the prior art. For example, mask processing may be performed in combination with a process of identifying a nipple region in a two-dimensional image described above. The processor may assign a value of 1 to a nipple region identified in each two-dimensional image and assign a value of 0 to remaining regions. And/or the nipple region may be adjusted to be white, and the remaining regions may be adjusted to be black, so as to achieve an effect of image segmentation. The foregoing process is merely an exemplary description, and is not limited in the present application.

Furthermore, as shown in FIG. 7, the plurality of nipple mask images 721 to 725 may also be processed to obtain a final nipple mask image 731. Therefore, the nipple region in the nipple mask image 731 can be determined as the nipple region in the volumetric ultrasound image. Such a configuration manner fully takes the nipple position in each coronal plane two-dimensional image into consideration for comprehensive evaluation, and can enable positioning of the nipple region in the volumetric ultrasound image to better conform to anatomical structure, thereby enabling positioning of the nipple marker to be as accurate as possible.

In some examples, the processing of the plurality of nipple mask images to obtain a final nipple mask image may include the following steps:

performing screening on the plurality of nipple mask images, wherein the screening is performed according to at least one of the size and the position of a nipple region in each nipple mask image;

determining an overlapping region of nipple regions in a plurality of nipple mask images obtained after screening; and

determining the final nipple mask image based on the overlapping region.

The screening process helps to exclude an abnormal two-dimensional image caused due to an algorithm error or an imaging factor, and avoid interference of the abnormal image with evaluation of an overall nipple region in the volumetric ultrasound image. Such screening may be performed according to morphological information of a nipple mask in the two-dimensional image, for example, at least one of the size or the position. Referring to FIG. 7, the size of the nipple region in the nipple mask image 721 is significantly greater than the size of a normal nipple/or sizes of nipple regions in other nipple mask images, and in this case, a computer algorithm can exclude the nipple mask image 721. And/or the position of the nipple region in the nipple mask image 725 is significantly deviated from positions of nipple regions in other nipple mask images, and the computer algorithm can also exclude the nipple mask image 725.

After the foregoing screening process, the overlapping region of nipple regions in the remaining nipple mask images can be determined, and the final nipple mask image is determined based on the overlapping region. The nipple mask image determined through the foregoing operations can represent the position of the overall nipple region in the depth direction of the volumetric ultrasound image as far as possible, thereby improving accuracy of nipple marker positioning.

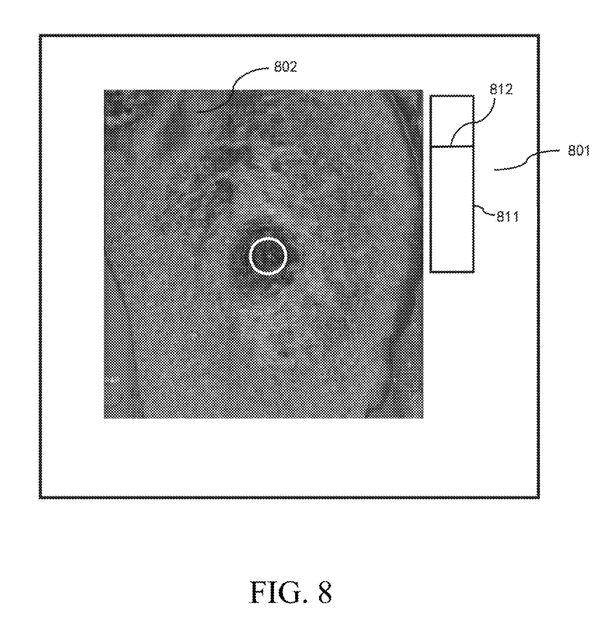

As described in the foregoing embodiments of the present application, the volumetric ultrasound image may include a plurality of two-dimensional images in the depth direction after undergoing a processing manner such as reconstruction. When being displayed, the plurality of two-dimensional images cannot be displayed simultaneously due to a viewing angle and the size of a display device. At least in view of these aspects, improvements are further provided in some embodiments of the present application. Referring to FIG. 8, FIG. 8 shows a schematic diagram of an ultrasound image including a depth marker 801 according to some embodiments of the present application.

In some embodiments, the depth marker 801 may be generated and displayed. The depth marker 801 indicates a depth corresponding to a current two-dimensional image 802 as described above, that is, the first depth.

As shown in FIG. 8, in an optional embodiment, the depth marker 801 may include a depth scale 811 and a depth indication line 812. An entire length of the depth scale 811 may be used to represent an entire depth range. A position where the depth indication line 812 is positioned represents the position of the current two-dimensional image 802 in the entire depth range. Therefore, by observing the depth marker 801, the user can quickly know the position of the current two-dimensional image 802 being displayed in the entire volumetric ultrasound image.

In an optional embodiment, the depth marker 801 can also be operated, for example, through a user input device such as a touchscreen or a trackball. Correspondingly, in response to being operated, the processor may move the depth marker 801 to indicate a second depth (not shown in the figure), and display a two-dimensional image at the second depth (not shown in the figure).

Such a configuration manner enables the user to quickly position different depths to browse a coronal plane two-dimensional image. In a possible case, if the two-dimensional image at the first depth positioned in the embodiments of the present application still does not meet the user's expectation, the user can check two-dimensional images across an entire depth by operating the depth marker so as to determine whether there is a problem in positioning of the nipple marker. If yes, appropriate measures can be taken, such as manually positioning the nipple marker.

Some embodiments of the present application further provide a breast ultrasound imaging system, including a processor. The processor is configured to perform the method described in any of the foregoing embodiments.

In some embodiments, the breast ultrasound imaging system further includes: an ultrasonic transducer, wherein under control of the processor, the ultrasonic transducer emits an ultrasonic beam toward the breast and receives an echo signal; and a display, wherein the display performs displaying under control of the processor.

It may be understood that, for components in the breast ultrasound imaging system, such as the ultrasonic transducer, the display, the processor, and the like, reference may be made to any of the foregoing embodiments in the present application, for example, reference may be made to any of the embodiments corresponding to FIGS. 1 and FIG. 2. In addition, for components not mentioned in the ultrasound imaging system, reference may also be made to any of the foregoing embodiments. The details thereof will not be further described herein.

An embodiment of the present application further proposes a non-transitory computer-readable storage medium, having computer program instructions stored thereon, wherein the computer program instructions, when executed by a processor, implement the steps of the method described in any of the foregoing embodiments in the present application.

An embodiment of the present disclosure further provides a computer program product, including computer-readable codes or a non-transitory computer-readable storage medium loaded with computer-readable codes, wherein when the computer-readable codes are run in a processor of a medical device, the processor in the medical device performs the steps of the foregoing method.

The computer-readable storage medium can be a tangible device that can hold and store instructions used by an instruction execution device. The computer-readable storage medium may be, for example, but is not limited to an electrical storage device, a magnetic storage device, an optical storage device, an electromagnetic storage device, a semiconductor memory device, or any suitable combination thereof. More specific examples (a non-exhaustive list) of the computer-readable storage medium include: a portable computer disk, a hard disk, a random access memory (RAM), a read only memory (ROM), a erasable programmable read only memory (EPROM or flash memory), a static random access memory (SRAM), a portable compact disc read-only memory (CD ROM), a digital versatile disk (DVD), a memory stick, a floppy disk, mechanical coding equipment, such as a punch card having instructions stored thereon or a structure of bumps within recessions, and any suitable combination thereof. The computer-readable storage medium used herein is not interpreted as transient signals themselves, such as radio waves or other freely propagated electromagnetic waves, electromagnetic waves propagated through a waveguide or other transmission media (e.g., light pulses passing through a fiber optic cable), or electrical signals transmitted through electric wires.

The computer-readable program instructions described herein may be downloaded from a computer-readable storage medium to various computing/processing devices or downloaded to an external computer or external storage device via a network such as the Internet, a local area network, a wide area network and/or a wireless network. The network may include copper transmission cables, fiber transmission, wireless transmission, routers, firewalls, switches, gateway computers, and/or edge servers. A network adapter card or a network interface in each computing/processing device receives computer-readable program instructions from the network and forwards the computer-readable program instructions, for storing them in a computer-readable storage medium in each computing/processing device.

Computer program instructions for executing the operations of the present disclosure can be assembly instructions, instruction set architecture (ISA) instructions, machine instructions, machine related instructions, microcode, firmware instructions, state setting data, or source code or object code written in any combination of one or more programming languages, the programming language including object oriented programming languages such as Smalltalk, C++ and the like, and conventional procedural programming languages such as the “C” language or similar programming languages. The computer-readable program instructions can be executed entirely or partly on a user computer, executed as a stand-alone software package, executed partly on a user computer and partly on a remote computer, or executed entirely on a remote computer or server. In the case of a remote computer, the remote computer may be connected to a user computer through any kind of network, including a local area network (LAN) or a wide area network (WAN), or connected to an external computer (for example, through the Internet, using an Internet service provider). In some embodiments, an electronic circuit, for example, a programmable logic circuit, a field-programmable gate array (FPGA), or a programmable logic array (PLA), may execute the computer-readable program instructions by utilizing state information of the computer-readable program instructions to personalize the electronic circuit, in order to implement various aspects of the present disclosure.

The aspects of the present disclosure are described herein with reference to the flowcharts and/or block diagrams of the methods, apparatuses (systems), and computer program products according to the embodiments of the present disclosure. It should be understood that each block of the flowcharts and/or block diagrams and combinations of various blocks in the flowcharts and/or block diagrams can be implemented by computer-readable program instructions.

These computer-readable program instructions may be provided to a processor of a general-purpose computer, a special-purpose computer, or other programmable data processing apparatuses, to produce a machine, so that these instructions, when executed by the processor of the computer or other programmable data processing apparatuses, produce an apparatus for implementing the functions/actions specified in one or more blocks of the flowcharts and/or block diagrams. Also, these computer-readable program instructions may be stored in a computer-readable storage medium. These instructions cause a computer, a programmable data processing apparatus, and/or other devices to work in a specific manner. Thus, the computer-readable medium storing the instructions includes an artifact, including instructions that implement various aspects of the functions/actions specified in one or more the flowcharts and/or block diagrams.

The computer-readable program instructions may also be loaded onto a computer, other programmable data processing apparatuses, or other devices, such that the computer, other programmable data processing apparatuses or other devices execute a series of operational steps, to generate a computer-implemented process, such that the functions/actions specified in one or more of the flowcharts and/or block diagrams are implemented by the instructions executed on the computer, other programmable data processing apparatuses, or other devices.

The flowcharts and block diagrams in the accompanying drawings illustrate system architectures, functions, and operations of possible implementations of the system, method, and computer program product according to a plurality of embodiments of the present disclosure. In this regard, each block in the flowcharts or block diagrams may represent a portion of a module, program segment, or instruction that contains one or more executable instructions for implementing the specified logical functions. In some alternative implementations, the functions denoted in the blocks can also occur in a different order than that illustrated in the drawings. For example, two consecutive blocks can actually be executed substantially in parallel, and sometimes can also be executed in a reverse order, depending upon the functions involved. It should also be noted that each block of the block diagrams and/or flowcharts, and combinations of blocks in the block diagrams and/or flowcharts can be implemented in a dedicated hardware-based system that executes the specified function or action, or can be implemented by a combination of dedicated hardware and computer instructions.

The purpose of providing the above specific embodiments is to allow the disclosure of the present application to be understood more thoroughly and comprehensively; however, the present application is not limited to said specific embodiments. A person skilled in the art should understand that various modifications, equivalent replacements, changes and the like can be further made to the present application and should be included in the scope of protection of the present application as long as these changes do not depart from the spirit of the present application.

1. A breast ultrasound imaging method, comprising:
acquiring a volumetric ultrasound image related to the breast, wherein the volumetric ultrasound image comprises a plurality of two-dimensional images in a depth direction;
identifying a nipple region in each two-dimensional image;
determining a nipple region in the volumetric ultrasound image and generating a corresponding nipple marker;
comparing the nipple region in the volumetric ultrasound image with the nipple region in each two-dimensional image, and automatically selecting a two-dimensional image at a first depth according to a comparison result; and
displaying the two-dimensional image at the first depth and the nipple marker simultaneously.

2. The method according to claim 1, wherein the nipple marker is applied to each two-dimensional image, and the position of the nipple marker in each two-dimensional image is consistent.

3. The method according to claim 1, wherein the acquisition of a volumetric ultrasound image of the breast comprises:
moving an ultrasonic transducer on a plane perpendicular to the depth direction while using the ultrasonic transducer to perform two-dimensional ultrasound imaging on the breast to obtain an image set related to the breast; and
performing synthesis processing on the image set to obtain the volumetric ultrasound image.

4. The method according to claim 1, wherein the plurality of two-dimensional images are acquired by segmenting the volumetric ultrasound image.

5. The method according to claim 1, further comprising:
generating and displaying a depth marker, wherein the depth marker indicates the first depth.

6. The method according to claim 5, further comprising:
in response to being operated, moving the depth marker to indicate a second depth, and displaying a two-dimensional image at the second depth.

7. The method according to claim 1, wherein
the comparison of the nipple region in the volumetric ultrasound image with the nipple region in each two-dimensional image comprises: respectively comparing the nipple region in each two-dimensional image with the nipple region in the volumetric ultrasound image to obtain similarity therebetween; and
the automatically selecting a two-dimensional image at a first depth according to a comparison result comprises: automatically selecting a two-dimensional image with high similarity as the two-dimensional image at the first depth according to a similarity comparison result.

8. The method according to claim 1, wherein the identification of a nipple region in each two-dimensional image is implemented by means of an artificial neural network.

9. The method according to claim 1, wherein the determination of a nipple region in the volumetric ultrasound image comprises:
performing mask processing on each two-dimensional image to obtain a plurality of nipple mask images;
processing the plurality of nipple mask images to obtain a final nipple mask image; and
determining a nipple region in the final nipple mask image as the nipple region in the volumetric ultrasound image.

10. The method according to claim 9, wherein the processing of the plurality of nipple mask images to obtain a final nipple mask image comprises:
performing screening on the plurality of nipple mask images, wherein the screening is performed according to at least one of the size and the position of a nipple region in each nipple mask image;
determining an overlapping region of nipple regions in a plurality of nipple mask images obtained after screening; and
determining the final nipple mask image based on the overlapping region.

11. A breast ultrasound imaging system, comprising:
a memory storing instructions; and
a processor configured to execute the instructions to:
acquire a volumetric ultrasound image related to the breast, wherein the volumetric ultrasound image comprises a plurality of two-dimensional images in a depth direction;
identify a nipple region in each two-dimensional image;
determine a nipple region in the volumetric ultrasound image and generating a corresponding nipple marker;
compare the nipple region in the volumetric ultrasound image with the nipple region in each two-dimensional image, and automatically selecting a two-dimensional image at a first depth according to a comparison result; and
display the two-dimensional image at the first depth and the nipple marker simultaneously.

12. The system according to claim 11, further comprising:
an ultrasonic transducer, wherein under control of the processor, the ultrasonic transducer emits an ultrasonic beam toward the breast and receives an echo signal; and
a display, wherein the display performs displaying under control of the processor.

13. A non-transitory computer-readable medium, wherein the non-transitory computer-readable medium has a computer program stored thereon, the computer program has at least one code segment, and the at least one code segment is executable by a machine to cause the machine to:
acquire a volumetric ultrasound image related to the breast, wherein the volumetric ultrasound image comprises a plurality of two-dimensional images in a depth direction;
identify a nipple region in each two-dimensional image;
determine a nipple region in the volumetric ultrasound image and generating a corresponding nipple marker;
compare the nipple region in the volumetric ultrasound image with the nipple region in each two-dimensional image, and automatically selecting a two-dimensional image at a first depth according to a comparison result; and
display the two-dimensional image at the first depth and the nipple marker simultaneously.
